(19) United States
(12) Patent Application Publication
(10) Pub. No.: US 2008/0175925 A1
(43) Pub. Date: Jul. 24, 2008

(54) Method and composition for the treatment of herpes virus

(76) Inventor: J. Craig Oxford, Nashville, TN (US)

Correspondence Address: Christina Ezell, STE B-10 PMB 200, 5133 Harding Rd., Nashville, TN, 37205, US

(21) Appl. No.: 12/009,597

(22) Filed: Jan. 19, 2008

Related U.S. Application Data
(60) Provisional application No. 60/897,019, filed on Jan. 23, 2007.

(51) Int. Cl. A61K 33/04; A61K 31/155; A61K 36/534; A61K 36/8962; A61P 31/22; A61K 36/00; A61K 38/06

(52) U.S. Cl. 424/702; 514/635; 424/747; 424/754; 424/725; 514/18

(57) ABSTRACT

A composition and method for oral application to mouth tissue to reduce the symptoms of herpes simplex virus infection, said composition comprising an anti-viral agent, an antioxidant agent, an analgesic agent and a flavorant.

This application claims the benefit of provisional patent application Ser. No. 60/897,019, filed Jan. 23, 2007 by the present inventor.

### FIELD OF THE INVENTION

The present invention relates to a preparation of a pharmaceutical oral application for topical application of a combination of antioxidants, anti-virals, analgesics and flavorants for the treatment of herpes simplex virus.

### BACKGROUND OF THE INVENTION

Oral herpes is an infection caused by the herpes simplex virus. The virus causes painful sores on lips, gums, tongue, roof of the mouth, and inside the cheeks. It also can cause symptoms such as fever and muscle aches. People contract herpes by touching infected saliva, mucous membranes, or skin. Because the virus is highly contagious, most people have been infected by at least 1 herpes subtype before adulthood.

After the herpes virus has infected an individual, it has a rather unique ability to proceed to 3 stages. In the primary infection the virus enters the skin or mucous membrane and reproduces. During this stage, oral sores and other symptoms, such as fever, may develop. However, the virus may not cause any sores and symptoms and many individuals do not know that they have the virus. This is called asymptomatic infection. Asymptomatic infection occurs twice as often as the disease with symptoms. During the latency stage the virus moves to a mass of nervous tissue in the spine called the dorsal root ganglion. There the virus reproduces again and becomes inactive. During the recurrence stage, the virus may reactivate and cause new sores and symptoms when an individual is exposed to certain emotional or physical stresses. Herpes simplex virus, type 1 HSV 1 causes 80% of cases of oral herpes infections. Herpes simplex virus, type 2 or HSV-2 causes the rest of the cases of oral herpes infections.

For oral herpes, the amount of time between contact with the virus and the appearance of symptoms, the incubation period, is 2-12 days. Most people average about 4 days. Signs and symptoms will last 2-3 weeks and include fever, tiredness, muscle aches, and irritability. Pain, burning, tingling, or itching occurs at the infection site before the sores appear and then clusters of blisters erupt. These blisters break down rapidly and, when seen, appear as tiny, shallow, gray ulcers on a red base. A few days later, they become crusted or scabbed and appear drier and more yellow.

The most intense pain caused by these sores occurs at the onset and make eating and drinking difficult. The sores may occur on the lips, the gums, the front of the tongue, the inside of the cheeks, the throat, and the roof of the mouth. They may also extend down the chin and neck. The gums may become mildly swollen and red and may bleed. Neck lymph nodes often swell and become painful. In people in their teens and 20s, herpes may cause a painful throat with shallow ulcers and a grayish coating on the tonsils.

Treatment for oral herpes includes medication for fever and taking plenty of fluids. A topical anesthetic such as viscous lidocaine (Dilocalne, Nervocaine, Xylocalne, Zilactin-L) may be prescribed to relieve pain. Oral or IV medication does exist for herpes but is not recommended for people with a normal immune system. It is used only for people with weakened immune systems, infants younger than 6 weeks, or people with severe disease.

One in five Americans experience cold sores, typically between one and three outbreaks a year. Current treatments include the topical treatments of docosanol (Abreva) and benzalkonium chloride (Viroxyn) as well as the pill and intravenous treatments of Acyclovir (Zovirax), Valacyclovir (Valtrex), and Famciclovir (Famvir).

Although there is no cure for herpesvirus infection, various supplements have shown the ability to reduce the severity and even the frequency of outbreaks. This might be due to their ability to support a healthy immune system (a weakened immune system has been associated with herpes reactivation). Antioxidants are particularly important immune-boosting supplements. Multiple clinical studies support the theory that antioxidants are of benefit in the management of herpesviruses. For example, Sheridan et. al. at the Ohio State University Health Sciences center used a cutaneous HSV-1 infection in mice to examine the ability of antioxidant components CRT to reduce lesion development, duration, and severity. Sheridan et. at., Antiviral Res. 1997, vol. 36, issue 3, pp. 157-66. CRT is a patented antioxidant formulation developed by Warner-Lambert Worldwide Consumer Healthcare R. and D. CRT contains three components that work synergistically: vitamin E, sodium pyruvate and membrane stabilizing fatty acids. SKH-1 male hairless mice were inoculated with 1×10(7) HSV-1 (McIntyre strain) on the dorsal surface of the mouse and treated with CRT formulations starting on the afternoon of the day of infection, and treated for the following 14 days. In the guinea pig model, the CRT formula that contained all three CRT components, worked synergistically to reduce lesion development, duration and severity scores significantly compared to vehicle control or acyclovir. Acyclovir was the only compound that reduced viral titers, but in contrast to CRT, acyclovir did not reduce lesion development, duration or severity. The quantitative effect of the three CRT components was demonstrated in the mouse model. Thus, antioxidants such as the CRT formulation are useful in the treatment of the sores caused by HSV-1.

### Chlorhexidine

Several studies have shown chlorhexidine to be an effective virucidal agent against strains of Herpesvirus hominis (HVH). For example, it has been demonstrated that a solution of 0.02% w/v chlorhexidine reduced the infectivity of two strains of HVH, namely the oral stain SUE (herpesvirus h simplex-1) and the genital strain MVC 70 (herpesvirus h simplex 0-2). Bailey et al., J Clin Path., 1972, vol. 25, pp. 76-78. Further, Libin in U.S. Pat. No. 5,855,872 discloses an ointment for treating diseased tissues resulting from HSV infection by synergistically combining two antimicrobial agents, the first agent being non-cationic and the other cationic. Libin discloses one possible cationic agent to be chlorhexidine.

More recently the in vitro effect of oral antiseptics containing 0.12-0.2% chlorhexidine on the McIntyre strain of herpersvirus h simplex-1 (HSV-1) was studied. Basqui et. al., J. Clin. Periodontal. 2001, vol. 28, issue 7, pp. 610-616. It was demonstrated that the undiluted chlorhexidine containing oral antiseptics had a significant antiviral on HSV-1 by complete inhibition of plaque formation on Vero Cell monolayers. Id. Based on these studies, it is likely that any rinse, ointment, cream, gel, or other formulation of an appropriate w/v % of chlorhexidine would be useful in the treatment of oral strains of HSV-1.

Chlorhexidine is also used to treat gingivitis. It helps to reduce the inflammation and swelling of gums and to reduce gum bleeding. Chlorhexidine is available only with a dentist's or medical doctor's prescription in the form of an oral rinse. Some commonly used brand names in the United States are Peridex and Perioguard; both contain 0.12% chlorhexidine gluconate. These solutions have a known bitter aftertaste and the impulse reaction to rinse with water not only decreases the solution's effectiveness but it increases the bitter taste.

### Peppermint Oil

Peppermint has high menthol content, and is often used as a flavoring in tea, ice cream, confectionery, chewing gum, and toothpaste. The oil also contains menthone and menthyl esters. It is the oldest and most popular flavor of mint-flavored confectionery. Peppermint can also be found in some shampoos and soaps, which give the hair a minty scent and produce a cooling sensation on the skin.

Peppermint, like many spices and herbs, is believed to have medicinal properties when consumed. It is known to help upset stomachs, to inhibit the growth of certain bacteria, and it can help smooth and relax muscles when inhaled or applied to the skin. Other health benefits are attributed to the high manganese, vitamin C and vitamin A content as well as trace amounts of various other nutrients such as fiber, iron, calcium, folate, potassium, tryptophan, magnesium, omega-3 fatty acids, riboflavin, and copper.

Peppermint is widely cultivated for its fragrant oil, which is obtained through steam distillation of the fresh aboveground parts of the plant. Peppermint oil has been used historically for numerous health conditions, including common cold symptoms, cramps, headache, indigestion, joint pain, and nausea. Peppermint leaf has been used for stomach/intestinal disorders and for gallbladder disease.

Peppermint oil is available in bulk herb oil, enteric-coated capsules, soft gelatin capsules, and in liquid form. In small doses such as in tea or chewing gum, peppermint is generally believed to be safe in healthy, non-pregnant, non-allergic adults. The United States is a principal producer of peppermint, and the largest markets for peppermint oil are manufacturers of chewing gum, toothpaste, mouthwash, and pharmaceuticals.

The virucidal effect of peppermint oil, the essential oil of Mentha piperita, against HSV-1 and HSV-2 has been demonstrated to be up to 99% inhibition in vitro in plaque reduction assays. Schuhmacher et. al., Phytomedicine, 2003, vol 10, pp. 504-510. It was determined that perppermint oil is capable of exerting a direct virucidal effect on HSV-1 and HSV-2 as both herpesviruses were significantly inhibited when herpes simplex virus was pretreated with peppermint oil prior to adsorption. Id. This study concluded that due to the lipophilic nature of the oil, which enables it to penetrate the skin, peppermint oil might be suitable for topical therapeutic use as a virucidal agent in recurrent herpes infection. Id.

### Garlic

Garlic is most often used as a seasoning or a condiment. When crushed or finely chopped it yields allicin, a powerful antibiotic and anti-fungal compound (phytoncide). It also contains alliin, ajoene, enzymes, vitamin B, minerals, and flavonoids. Garlic has substantial antiviral activity. Fresh garlic extract, in which thiosulfinates are the active components, has shown virucidal against every virus tested, including HSV1 and HSV2. Weber et. al., Planta Med. 1992, vol. 58, issue 5, pp. 417-23. The predominant thiosulfinate in fresh garlic extract is allicin. Allicin can be used for medicinal purposes as an antiviral agent.

### Clove Oil

Clove oil, is an essential oil from the clove plant. It is a natural analgesic used primarily in dentistry for its active ingredient eugenol. It can also be purchased in pharmacies over the counter, as a local anesthetic for dental pain relief, mainly toothache; it is also often found in the aromatherapy section of health food stores. The oil produced by cloves can be used in many things from flavoring medicine to remedies for bronchitis, the common cold, a cough, fever, sore throat and tending to infections. Its country of origin is India.

While clove oil is considered by many in the health profession to be an effective antibiotic, antiviral, analgesic, antioxidant, carminative, expectorant, antifungal, anti-parasitic, spasmodic, and stimulant, it is known best for its anesthetic properties. It is widely reported to be effective, and prior to the availability of safe, approved topical anesthetic drugs, was used by some dentists. It is considered safe in very small quantities (<1500 p.p.m.) as a food additive. Bruneton, J (1995). Pharmacognosy, Phytochemistry, Medicinal Plants. Hampshire, U.K.: Intercept Ltd. It is an intense oil that is antiseptic, carminative, warming, and very aromatic. It is often used as a flavoring in toothpaste, mouthwashes, and exotic foods.

### Glutathione

Glutathione, 2-amino-5-{[2-[(carboxymethyl)amino]-1-(mercaptomethyl)-2-oxoethyl]amino}-5-oxopentanoic acid, is γ-glutamylcysteinylglycine, a tripeptide. It contains an unusual peptide linkage between the amine group of cysteine and the carboxyl group of the glutamate side chain. Glutathione, an antioxidant, protects cells from toxins such as free radicals.

Glutathione is the principal intracellular non protein thiol and plays a major role in the maintenance of the intracellular redox state. It may be thought of as an intracellular redox buffer. Glutathione is a nucleophilic scavenger and an electron donor via the sulflhydryl group of its business residue, cysteine. Its reducing ability maintains molecules such as ascorbate and proteins in their reduced state. Glutathione is also the cofactor for the selenium-containing glutathione peroxidases (see Selenium, infra), which are major antioxidant enzymes. These enzymes detoxify peroxides, such as hydrogen peroxide and other peroxides. Another antioxidant activity of glutathione is the maintenance of the antioxidant/reducing agent ascorbate in its reduced state. This is accomplished via glutathione-dependent dehydroascorbate reductase which is comprised of glutaredoxin and protein isomerase reductase. Glutathione may also react with the reactive nitrogen species peroxynitrite to form S-nitrosoglutathione.

Glutathione S-transferases (GSTs) consist of a family of multifunctional enzymes that metabolize a wide variety of electrophilic compounds via glutathione conjunction. GSTs are involved in the detoxification of xenobiotic compounds and in the protection against such degenerative diseases as cancer. The mechanism of these enzymes involves a nucleophilic attack by glutathione on an electrophilic substrate. The resulting glutathione conjugates that form are more soluble than the original substrates and thus more easily exported from the cell. The release of glutathione-S-conjugates from cells is an ATP-dependent process mediated by membrane glycoproteins belonging to the multidrug-resistance protein (MRP) family. Proteins of the MRP family are essential for the transport of glutathione S-conjugates into the extracellular space. They are also known as glutathionine-5-conjugate pumps.

There is evidence for antiviral activity of glutathione with in vitro inhibition of HSV-1 replication. The role of reduced glutathione (GSH) in the in vitro infection and replication of HSV-1 was investigated. Intracellular endogenous GSH levels dramatically decreased in the first 24 h after virus adsorption, starting immediately after virus challenge. The addition of exogenous GSH was not only able to restore its intracellular levels almost up to those found in uninfected cells, but also to inhibit >99% the replication of HSV-1. The data suggests that exogenous GSH inhibits the replication of HSV-1 by interfering with very late stages of the virus life cycle, without affecting cellular metabolism. Antiviral Res., 1995 vol 27, issue 3, pp. 237-53.

Hersh in U.S. Pat. No. 6,228,347 discloses a gel, paste, or lozenge for oral application to prevent gum disease containing glutathione, selenium, flavorants, and an abrasive. The formulation is said to reduce the reactive oxygen and other free radical species which are causative inflammatory factors in establishing and promoting gingival diseases. The present invention is an improvement on such a formulation as it contemplates the use of an antiviral, antioxidant, and analgesic oral application for the treatment of herpes simplex virus.

### Selenium

Selenium is a trace mineral that is essential to good health but required only in small amounts. Selenium is incorporated into proteins to make selenoproteins, which are important antioxidant enzymes. The antioxidant properties of selenoproteins help prevent cellular damage from free radicals. Free radicals are natural by-products of oxygen metabolism that may contribute to the development of chronic diseases such as cancer and heart disease. Although it is toxic in large doses, selenium is an essential micronutrient in all known forms of life. It is a component of the unusual amino acids selenocysteine and selenomethionine. In humans, selenium is a trace element nutrient which functions as cofactor for reduction of antioxidant enzymes such as glutathione peroxidases and thioredoxin reductase. It also plays a role in the functioning of the thyroid gland by participating as a cofactor for thyroid hormone deiodinases.

The antioxidant activity of selenium is mainly accounted for by virtue of its role in the formation and function of the selenium-dependent glutathione peroxidases (GSHPx). Glutathione peroxidases use reducing equivalents from glutathione to detoxify hydroperoxides. There are four different glutathione peroxidases. GSHPx-1 is present in most cells of the body. GSHPx-2 (originally known as GSHPx-GI) is mainly found in the cells of the gastrointestinal tract. GSHPx-3 is an extracellular glutathione peroxidase. GSHPx-4 is a membrane-bound hydroperoxide glutathione peroxidase. GSHPx-4 is also known as phospholipid hydroperoxide or PHGPx. GSHPx-4 can detoxify phospholipid hydroperoxides and, along with d-alpha-tocopherol, helps prevent oxidative damage to membranes. GSHPx-3, the extracellular glutathione peroxidase, eliminates peroxides in the extracellular fluid.

Glutathione peroxidases detoxify hydrogen peroxide and fatty acid-derived hydroperoxides. This is the antioxidant role of these enzymes. However, recent research indicates that reactive oxygen species play important roles in signal transduction processes. Therefore, by affecting the concentrations of reactive oxygen species in cells, the glutathione peroxidases may also be considered to play regulatory roles in signal transduction.

Antioxidant activity of selenium can also be accounted for by its role in the selenium-dependent thioredoxin reductases. These enzymes reduce intramolecular disulfide bonds and regenerate ascorbic acid from dehydroascorbic acid. Thioredoxin reductases can also affect the redox regulation of a variety of factors, including ribonucleotide reductase (the enzyme that converts ribonucleoside diphosphates to deoxyribonucleoside diphosphates), the glucocorticoid receptor and the transcription factors AP-1 and NF-KappaB.

### Vitamin C

The term vitamin C applies to substances that possess antiscorbutic activity and includes two compounds and their salts: L-ascorbic acid, commonly called ascorbic acid, and L-dehydroascorbic acid. Ascorbic acid is the major dietary form of vitamin C. The terms vitamin C, ascorbic acid and ascorbate are commonly used interchangeably.

Vitamin C has antioxidant activity. It may also have anti-atherogenic, anticarcinogenic, antihypertensive, antiviral, antihistaminic, immunomodulatory, opthalmoprotective and airway-protective actions. Vitamin C may aid in the detoxification of some heavy metals, such as lead and other toxic chemicals. Vitamin C is arguably the most important water-soluble biological antioxidant. It can scavenge both reactive oxygen species and reactive nitrogen species. Ascorbic acid or, more specifically, ascorbate is an excellent reducing agent, and it acts as a cofactor in various biochemical reactions to reduce the transition metals, iron and copper.

Ascorbate can be oxidized by most reactive oxygen and nitrogen species thought to play roles in tissue injury associated with various diseases. These species include superoxide, hydroxyl, peroxyl and nitroxide radicals, as well as such non-radical reactive species as singlet oxygen, peroxynitrite and hypochlorite. By virtue of this scavenging activity, ascorbate inhibits lipid peroxidation, oxidative DNA damage and oxidative protein damage. Ascorbate is oxidized by reactive oxygen and nitrogen species to the semidehydroascorbate radical that is either reconverted to ascorbate via the enzyme NADH semidehydroascorbate reductase or is converted to dehydroascorbate. Dehydroascorbate in turn can be converted back to ascorbate via glutathione-dependent enzymes or catabolized.

Ascorbate can act as a secondary antioxidant. At least in vitro, ascorbate regenerates the major lipid antioxidant alpha-tocopherol from the alpha-tocopheroxyl radical form. Ascorbate may also participate in regenerating and sparing alpha-tocopherol in vivo, though this has not been clearly demonstrated. Vitamin C helps preserve intracellular reduced glutathione concentrations. This activity likely helps maintain nitric oxide levels and potentiates its vasoactive effects. Oral vitamin C can reach high enough concentrations intracellularly to scavenge superoxide radicals. Thus, intracellular sources of superoxide that impair nitric oxide may be scavenged by oral vitamin C. Recently, it has been found that ascorbic acid enhances nitric oxide synthase activity by increasing intracellular tetrahydrobiopterin.

### Parsley Oil

Parsley is a rich source of vitamin C and yields a fixed oil, an essential oil, and tannins. The seeds contain both a fixed and volatile oil, the latter being comprised of apiol, myristicin, tetramethoxybenzene, pinene, and other compounds. The leaf or herb oil is considered superior to seed oil, as the volatile characteristics are more similar to parsley leaves. The fixed oil of parsley contains petroseline plus oleic, linoleic, palmatic, and other fatty acids.

The seeds, leaves, and essential oils of parsley are utilized as condiments or seasonings. Fresh leaves are used for garnishing such food dishes as meat, fish, and vegetables. Fresh, dried, and dehydrated leaves flavor a wide array of food products, including salads, sauces, soups, stews, eggs, and processed foods. Parsley-seed oil is employed as a fragrance in perfumes, soaps, and creams.

It is well known for its use in eliminating garlic from the breath. A large number of sulfur compounds contribute to the smell and taste of garlic. Diallyl disulfide is believed to be an important odor component of garlic. Allicin has been found to be the compound most responsible for the spiciness of raw garlic. Parsley is edible and so is the oil produced from it. It has a mild flavor and because parsley has a high chlorophyll content, it is a natural breath sweetener. Most garlic pills on the market are combined with parsley. Glutathione, as mentioned earlier, is also a sulfur containing compound whose odor can also be successfully treated with parsley oil.

### Local Anesthetics

Many over the counter (OTC) oral herpes treatments contain a local anesthetic such as benzocaine or lidocaine. Benzocaine is an ester, a compound made from the organic acid PABA (para-aminobenzoic acid) and ethanol. Pain is caused by the stimulation of nerve endings. When the nerve endings are stimulated, sodium enters the nerve ending, which causes an electrical signal to build up in the nerve. Once the electrical signal becomes big enough, it is able to travel to the brain, which then interprets this as pain. Esters of PABA work as a chemical barrier, stopping the sodium from being able to enter the nerve ending. Lidocaine, the first amino amide-type local anesthetic, alters depolarization in neurons, by blocking the fast sodium (Na+) channels in the cell membrane. With sufficient blockade, the membrane will not depolarize and so not transmit an action potential, leading to its anesthetic effects. While both are effective anesthetics, a small percentage of the patient population has an allergic response to these types of topical anesthetics.

Doshi et. al. in U.S. Pat. No. 6,365,131 discloses a pharmacuetical dental gel formation containing the bacterialcidal metronidazole benzoate, the virucidal chlorhexidine glucontate, and a local anesthetic for the treatment of gingivitis and periodontis. The present invention is an improvement on such a formulation as it contemplates the use of antioxidants including glutathione in conjunction with the virucidal chlorhexidine and an analgesic. Glutathione serves not only in an antioxidant capacity, but as referenced supra, there is significant evidence of antiviral activity of gluathione with in vitro inhibition of HSV-1 replication not disclosed in Doshi patent.

### SUMMARY OF THE INVENTION

An ideal treatment of oral herpes sore (known as cold sores) would be in the form of a mouth rinse, gel, patch, or other suitable oral application. The ideal treatment would be antiviral, analgesic and antioxidant in nature. The present invention seeks to combine the antiviral activities of chlorhexidine, peppermint oil, and garlic; the analgesic properties of topical anesthetics or natural anesthetic of clove oil; and the antioxidant properties of glutathione, selenium, and vitamin C or other suitable antioxidant compounds. A formulation containing clove oil instead of a topical anesthetic would be preferable to the patient population currently allergic to topical anesthetics such as lidocaine or benzocaine. Additionally, parsley oil is used to eliminate the foul odor and taste from the sulfur containing compounds of glutathione and garlic as well as eliminate the bitter taste of chlorhexidine. The combination of an antiviral such as chlorhexidine, peppermint oil, garlic; an anesthetic such as benzocaine, lidocaine or clove oil, an antioxidant such as glutathione, selenium, vitamin C; and parsley oil is formulated as a mouth rinse, gel, patch, or other suitable oral application. The present invention represents a novel approach to cold sore treatment and management with significant commercial implications as a high percentage of the population experiences cold sores on a reoccurring basis.

### DETAILED DESCRIPTION OF THE INVENTION

The present invention involves a composition and method in accordance with the invention for treating a herpes simplex virus (HSV) infection is preferably in the form of a mouth rinse, gel, patch or other suitable oral application, making it possible to coat the diseased tissue to bring the active ingredients of the oral application in contact with the tissue. The present composition comprises the active ingredients of an anti-viral agent, an antioxidant agent, and an analgesic agent. The present method for reducing the symptoms of herpes simplex virus involves the application of the above-mentioned composition to diseased mouth tissues in the form of a gel, patch, mouth rinse or other suitable oral application.

The anti-viral agent can be a source of chlorhexidine, garlic, peppermint oil, a source of glutathione or any combination of these anti-viral agents. In a preferred embodiment, the main anti-viral active ingredient is a chlorhexidine solution in an amount of about 0.12% to 0.20% by weight. The further addition of garlic, peppermint oil, and glutathione serves to supplement the anti-viral activity of chlorhexidine.

The antioxidant agent can be a source of glutathione, selenium, vitamin C or any combination of these antioxidants. In a preferred embodiment, a source of glutathione, reduced glutathione (GSH) is used in conjunction with a source of selenium and a source of vitamin C as glutathione must act in combination with other cellular antioxidants in order to be reduced so that it may renew its role as a free radical scavenger. Selenium compounds work with reduced glutathione through glutathione peroxidase. Gluthathione and selenium act synergistically. The source of selenium includes elemental selenium and selanoamino acids including selenomethionine and selenocysteine. L-ascorbic acid (vitamin C) or its derivatives can be employed in these compositions primarily for their antioxidant activities. Stabilized vitamin C is employed so that it does not lose it physiological reducing activities because of its high susceptibility to oxidation.

The analgesic agent can be a source of manufactured topical anesthetic such as lidocaine, benzocaine, or other suitable anesthetic or can be a source of natural anesthetic such as clove oil. The use of clove oil is particularly useful for those patients allergic to traditional manufactured topical anesthetics.

The present composition may include flavorings. Flavors may be based on peppermint oil, parsley, clove oil or a combination of the flavors. Parsley oil is used especially useful to eliminate the foul odor and taste from the sulfur containing compounds of glutathione and garlic as well as eliminate the bitter taste of chlorhexidine.

1. A composition for oral application to mouth tissue to reduce the symptoms of herpes simplex virus, said composition comprising an anti-viral agent, an antioxidant agent, and an analgesic agent.

2. A composition in accordance with claim 1, wherein said anti-viral agent is selected from the group consisting of a source of chlorhexidine, garlic, peppermint oil, a source of glutathione and combinations thereof.

3. A composition in accordance with claim 1, wherein said antioxidant agent is selected from the group consisting of a source of glutathione, a source of selenium, a source of vitamin C and combinations thereof.

4. A composition in accordance with claim 1, wherein said analgesic agent is selected from the group consisting of lidocaine, benzocaine, clove oil and combinations thereof.

5. A composition in accordance with claim 1 further comprising a flavorant.

6. A composition in accordance with claim 5, wherein said flavorant agent is selected from the group consisting of peppermint oil, parsley, clove oil and combinations thereof.

7. A method of reducing symptoms of herpes simplex virus, said method comprising applying to human mouth tissue a composition comprising an anti-viral agent, an antioxidant agent, and an analgesic agent.

8. A method in accordance with claim 7, wherein said anti-viral agent is selected from the group consisting of a source of chlorhexidine, garlic, peppermint oil, a source of glutathione and combinations thereof.

9. A method in accordance with claim 7, wherein said antioxidant agent is selected from the group consisting of a source of glutathione, a source of selenium, a source of vitamin C and combinations thereof.

10. A method in accordance with claim 7, wherein said analgesic agent is selected from the group consisting of lidocaine, benzocaine, clove oil and combinations thereof.

11. A method in accordance with claim 7, wherein said composition further comprises a flavorant.

12. A method in accordance with claim 11, wherein said flavorant agent is selected from the group consisting of peppermint oil, parsley, clove oil and combinations thereof.

13. A composition in accordance with claim 2, wherein said source of chlorhexidine comprised of chlorhexidine gluconate present in a range of about 0.12% to about 0.20% by weight based on the total weight of said composition.

14. A composition in accordance with claim 3, wherein said source of selenium comprises a member selected from the group consisting of elemental selenium and a selenoamino acid.

15. A composition in accordance with claim 3, wherein said source of glutathione is reduced glutathione (GSH).

16. A composition in accordance with claim 14, wherein said selanoamino acid comprises a member selected from the group consisting of selenomethionine and selenocysteine.

17. A composition in accordance with claim 3 wherein said source of vitamin C comprises a member selected from the group consisting of ascorbic acid and a derivative of ascorbic acid.

18. A method in accordance with claim 8, wherein said source of chlorhexidine comprises chlorhexidine gluconate present in a range of about 0.12% to about 0.20% by weight based on the total weight of said composition.

19. A method in accordance with claim 9, wherein said source of selenium comprises a member selected from the group consisting of elemental selenium and a selenoamino acid.

20. A method in accordance with claim 19, wherein said selanoamino acid comprises a member selected from the group consisting of selenomethionine and selenocysteine.

21. A method in accordance with claim 9, wherein said source of vitamin C comprises a member selected from the group consisting of ascorbic acid and a derivative of ascorbic acid.
